(19) United States
(12) Patent Application Publication
(10) Pub. No.: US 2025/0129415 A1
(43) Pub. Date: Apr. 24, 2025

(54) POLYNUCLEOTIDE SEQUENCING USING IONOPHORES

(71) Applicant: Illumina, Inc., San Diego, CA (US)

(72) Inventors: Jeffrey Mandell, Rancho Santa Fe, CA (US); Boyan Boyanov, San Diego, CA (US)

(73) Assignee: Illumina, Inc., San Diego, CA (US)

(21) Appl. No.: 18/565,027

(22) PCT Filed: Jun. 30, 2022
(86) PCT No.: PCT/US2022/035797

§ 371 Date: Nov. 28, 2023

Related U.S. Application Data
(60) Provisional application No. 63/218,130, filed on Jul. 2, 2021.

(51) Int. Cl. C12Q 1/6869; G01N 27/12

(52) U.S. Cl. CPC C12Q 1/6869; G01N 27/12

(57) ABSTRACT

Polynucleotide sequencing using ionophores is provided herein. In some examples, a sequencing method includes inhibiting current flow across a barrier; contacting a polymerase with a first polynucleotide, a second polynucleotide, and a fluid comprising a first nucleotide coupled to a first ionophore; while the polymerase adds the first nucleotide to the second polynucleotide based on a sequence of the first polynucleotide, providing a first current flow across the barrier using the first ionophore; and identifying the first nucleotide using an electrical characteristic of the first ionophore.

### CROSS-REFERENCE TO RELATED APPLICATION

This application claims the benefit of U.S. Provisional Patent Application No. 63/218,130, filed on Jul. 2, 2021, and entitled “POLYNUCLEOTIDE SEQUENCING USING IONOPHORES”, the entire contents of which are incorporated by reference herein.

### FIELD

This application relates to methods of polynucleotide sequencing.

### SEQUENCE LISTING

The instant application contains a Sequence Listing which has been submitted electronically in ASCII format and is hereby incorporated by reference in its entirety. Said ASCII copy, created on Jun. 28, 2022, is named IP-2076-PCT_SL and is 1,152 bytes in size.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in a α-hemolysin nanopore. Or, for example, KF-DNA complexes have been distinguished on the basis of their properties when captured in an electric field atop an α-hemolysin nanopore. In still another example, nucleic acid sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a growing nucleic acid. The charge blockade label is detected by the nanopore to determine the presence and identity of the incorporated nucleotide and thereby determine the sequence of a template nucleic acid. In still other examples, constructs include a transmembrane protein pore subunit and a nucleic acid handling enzyme.

However, such previously known compositions, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved compositions, systems, and methods for sequencing polynucleotides.

### SUMMARY

Polynucleotide sequencing using ionophores is provided herein.

Some examples herein provide a nucleotide analogue. The nucleotide analogue may include a sugar; a nucleobase coupled to the sugar; a phosphate group coupled to the sugar; and an ionophore indirectly coupled to the sugar via the phosphate group.

In some examples, the ionophore includes gramicidin A, gramicidin B, gramicidin C, or fengycin.

Additionally, or alternatively, in some examples, the ionophore selectively passes anions. Alternatively, in some examples, the ionophore selectively passes cations.

Additionally, or alternatively, in some examples, the ionophore is coupled to the phosphate group via a linker.

Additionally, or alternatively, in some examples, the phosphate group is selected from the group consisting of triphosphate, tetraphosphate, pentaphosphate, and hexaphosphate.

Some examples herein provide a fluid including a plurality of nucleotide analogues. Each of the nucleotide analogues may include a sugar; a nucleobase coupled to the sugar; a phosphate group coupled to the sugar; and an ionophore indirectly coupled to the sugar via the phosphate group. The ionophores of a first set of the nucleotide analogues may be different than the ionophores of a second set of the nucleotide analogues.

In some examples, the ionophores include gramicidin A, gramicidin B, gramicidin C, or fengycin.

Additionally, or alternatively, in some examples, the ionophores of the first set of nucleotide analogues have a first electrical characteristic, and the ionophores of the second set of the nucleotide analogues have a second electrical characteristic that is different than the first electrical characteristic.

Additionally, or alternatively, in some examples, the ionophores of a third set of nucleotide analogues have a third electrical characteristic that is different than the first and second electrical characteristics. Additionally, or alternatively, in some examples, the ionophores of a fourth set of the nucleotide analogues have a fourth electrical characteristic that is different than the first, second, and third electrical characteristics.

Additionally, or alternatively, in some examples, the first, second, third, or fourth electrical characteristic includes a magnitude of a current, resistance, or voltage through the respective ionophore.

Additionally, or alternatively, in some examples, the first, second, third, or fourth electrical characteristic includes a temporal duration of a current through the respective ionophore.

Some examples herein provide a sequencing method. The sequencing method may include inhibiting current flow across a barrier. The sequencing method may include contacting a polymerase with a first polynucleotide, a second polynucleotide, and a fluid including a first nucleotide coupled to a first ionophore. The sequencing method may include, while the polymerase adds the first nucleotide to the second polynucleotide based on a sequence of the first polynucleotide, providing a first current flow across the barrier using the first ionophore. The sequencing method may include identifying the first nucleotide using an electrical characteristic of the first ionophore.

In some examples, the electrical characteristic includes a magnitude of the first current flow, a resistance through the first ionophore, or a voltage through the first ionophore.

Additionally, or alternatively, in some examples, the electrical characteristic includes a temporal duration of the first current flow.

Additionally, or alternatively, in some examples, the polymerase is coupled to the barrier.

Additionally, or alternatively, in some examples, the method further includes decoupling, using the polymerase, the first ionophore from the first nucleotide; and diffusing the decoupled first ionophore away from the barrier so as to inhibit current flow across the barrier.

Additionally, or alternatively, in some examples, the barrier includes a second ionophore to which the first ionophore becomes coupled to provide the first current flow across the barrier.

In some examples, the polymerase is coupled to the second ionophore.

Additionally, or alternatively, in some examples, the method further includes decoupling the first ionophore from the second ionophore so as to inhibit current flow across the barrier again.

Additionally, or alternatively, in some examples, the first and second ionophores are selected from the group consisting of gramicidin A, gramicidin B, gramicidin C, and fengycin.

Additionally, or alternatively, in some examples, the barrier includes a plurality of second ionophores.

Additionally, or alternatively, in some examples, the first ionophore selectively passes anions. Alternatively, in some examples, the first ionophore selectively passes cations.

Additionally, or alternatively, in some examples, the first ionophore does not pass polynucleotides.

Additionally, or alternatively, in some examples, the barrier includes an electrical insulator through which the first ionophore forms an aperture.

Additionally, or alternatively, in some examples, the barrier includes a membrane. In some examples, the membrane includes a lipid bilayer. In some examples, the first ionophore inserts into a layer of the lipid bilayer.

Additionally, or alternatively, in some examples, the fluid further includes a plurality of additional nucleotides each coupled to a respective ionophore. The method further may include, while the polymerase sequentially adds the each of the additional nucleotides to the second polynucleotide based on a sequence of the first polynucleotide, sequentially providing additional current flows across the barrier using the respective ionophore. The method further may include identifying the additional nucleotides using additional electrical characteristics of the additional ionophores.

In some examples, at least some of the respective ionophores have different modifications than one another.

Additionally, or alternatively, in some examples, the additional electrical characteristic includes a magnitude of the additional current flow, a resistance through the respective ionophore, or a voltage through the respective ionophore.

Additionally, or alternatively, in some examples, the additional electrical characteristic includes a temporal duration of the additional current flow through the respective ionophore.

Additionally, or alternatively, in some examples, the barrier includes a second ionophore to which each of the respective ionophores become coupled to respectively provide the additional current flows across the barrier.

Additionally, or alternatively, in some examples, the barrier includes a plurality of second ionophores to which the respective ionophores selectively become coupled.

Additionally, or alternatively, in some examples, identifying the first nucleotide includes transferring to an electrode, using a redox reaction, electrons from ions passed by the first ionophore. In some examples, the ions include Cl−.

Additionally, or alternatively, in some examples, identifying the first nucleotide includes applying a first electric field to generate, at a first electrode, a first aggregation of ions passed by the first ionophore; and using the first aggregation of ions to generate a first transient current through an external circuit. In some examples, the ions include potassium (K+) or sodium (Na+). Additionally, or alternatively, in some examples, the method further includes applying a second electric field to generate, at a second electrode opposite the first electrode, a second aggregation of ions passed by the first ionophore; and using the second aggregation of ions to generate a second transient current through the external circuit. In some examples, the method further includes repeatedly applying the first and second electric fields to repeatedly generate the first and second transient currents.

Some examples herein provide a composition. The composition may include a barrier. The composition may include a polymerase in contact with a first polynucleotide, a second polynucleotide, and a fluid including a first nucleotide coupled to a first ionophore. The polymerase may add the first nucleotide to the second polynucleotide based on a sequence of the first polynucleotide. The first ionophore may provide a first current flow across the barrier.

In some examples, the polymerase is coupled to the barrier.

Additionally, or alternatively, in some examples, the first ionophore is detachable, by the polymerase, from the first nucleotide so as to diffuse away from the barrier so as to inhibit current flow across the barrier.

Additionally, or alternatively, in some examples, the barrier includes a second ionophore to which the first ionophore is coupled. In some examples, the first ionophore is detachable from the second ionophore so as to inhibit current flow across the barrier.

Additionally, or alternatively, in some examples, the first and second ionophores are selected from the group consisting of gramicidin A, gramicidin B, gramicidin C, and fengycin.

Additionally, or alternatively, in some examples, the polymerase is coupled to the barrier.

Additionally, or alternatively, in some examples, the barrier includes a plurality of second ionophores.

Additionally, or alternatively, in some examples, the first ionophore selectively passes anions. Alternatively, in some examples, the first ionophore selectively passes cations.

Additionally, or alternatively, in some examples, the first ionophore does not pass polynucleotides.

Additionally, or alternatively, in some examples, the barrier includes an electrical insulator through which the first ionophore forms an aperture.

Additionally, or alternatively, in some examples, the barrier includes a membrane. In some examples, the membrane includes a lipid bilayer. In some examples, the first ionophore inserts into a layer of the lipid bilayer.

Additionally, or alternatively, in some examples, the fluid further includes a plurality of additional nucleotides each coupled to a respective ionophore. In some examples, at least some of the respective ionophores have different modifications than one another. Additionally, or alternatively, in some examples, the membrane includes a second ionophore to which each of the respective ionophores is couplable.

Additionally, or alternatively, in some examples, the barrier includes a plurality of second ionophores to which the respective ionophores are selectively couplable.

Some examples herein provide a system. The system may include any of the foregoing compositions, and electrical circuitry for identifying the first nucleotide using an electrical characteristic of the first ionophore.

In some examples, the electrical circuitry is for measuring a magnitude of the first current flow, a resistance through the first ionophore, or a voltage through the first ionophore.

Additionally, or alternatively, in some examples, the electrical circuitry is for measuring barrier temporal duration of the first current flow.

Additionally, or alternatively, in some examples, the electrical circuitry is for transferring to an electrode, using a redox reaction, electrons from ions passed by the first ionophore. In some examples, the ions include Cl−.

Additionally, or alternatively, in some examples, the electrical circuitry is for applying a first electric field to generate, at a first electrode, a first aggregation of ions passed by the first ionophore, and the first aggregation of ions generates a first transient current through the electrical circuitry. In some examples, the ions include potassium (K+) or sodium (Na+). Additionally, or alternatively, in some examples, the electrical circuitry is for applying a second electric field to generate, at a second electrode, a second aggregation of ions passed by the first ionophore, and the second aggregation of ions generates a second transient current through the electrical circuitry. In some examples, the electrical circuitry is for repeatedly applying the first and second electric fields to repeatedly generate the first and second transient currents.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1E schematically illustrate example compositions and systems for polynucleotide sequencing using ionophores.

FIG. 2 schematically illustrates example signals that may be obtained during polynucleotide sequencing using the system of FIGS. 1A-1E.

FIG. 3 schematically illustrates an alternative example composition and system for polynucleotide sequencing using ionophores.

FIG. 4 schematically illustrates another alternative example composition and system for polynucleotide sequencing using ionophores.

FIG. 5 schematically illustrates another alternative example composition and system for polynucleotide sequencing using ionophores.

FIG. 6 illustrates a flow of operations in an example method for polynucleotide sequencing using ionophores.

### DETAILED DESCRIPTION

Polynucleotide sequencing using ionophores is provided herein.

For example, the present disclosure provides compositions, systems, and methods for polynucleotide sequencing that use ionophores, which are relatively simple compounds that may insert into barriers, such as membranes, in such a manner as to permit ions to flow across the membrane under the bias of an external electric field, while inhibiting the flow of uncharged molecules, such as nucleotides, and also inhibiting the flow of relatively large molecules such as polynucleotides. The ionophores respectively may be coupled to nucleotides that are being used in a sequencing-by-synthesis (SBS) process. For example, a polymerase sequentially may add the nucleotides to a first polynucleotide based on the sequence of a second polynucleotide for which it is desired to determine the sequence. As the polymerase sequentially is acting upon those nucleotides, the ionophores respectively coupled to those nucleotides may become coupled to a barrier and may provide a measurable current flow, caused by selective ion conduction, across that barrier. The barrier substantially may not conduct any current flow in the absence of an ionophore. Therefore, the presence of the current flow may be interpreted as meaning that an ionophore is coupled to the barrier, and that therefore a nucleotide is being acted upon by the polymerase. The presence of the ionophore coupled the barrier may be electrically characterized in any suitable manner, e.g., a magnitude or temporal duration of current, resistance, or voltage, and from such characterization the nucleotide may be identified.

Furthermore, ionophores having different ion conduction characteristics than one another respectively may be coupled to different nucleotides, and as such a given nucleotide may be identified based on the particular electrical characteristic of the ionophore to which that nucleotide is coupled. As the polymerase acts upon each nucleotide, respective currents across the membrane may be provided by the corresponding ionophores. The presence of the respective ionophores coupled the barrier may be electrically characterized in any suitable manner, e.g., a magnitude or temporal duration of current, resistance, or voltage, and from such characterization the respective nucleotide may be identified. As such, the second polynucleotide may be sequenced without the need for fluidically regulating the SBS process. After the polymerase adds each nucleotide to the first polynucleotide, the polymerase may cleave the corresponding ionophore from that nucleotide, following which the ionophore may diffuse away from the barrier such that the current flow returns to zero. As such, in various examples, the present compositions, systems, and methods are compatible with single-pot processing, electrical-based detection of labeled nucleotides, relatively high-density flow cells, and as such may provide for relatively inexpensive sequencing instruments using relatively inexpensive consumables.

First, some terms used herein will be briefly explained. Then, some example compositions, example methods, and example systems including electrical detection circuitry that can be used for polynucleotide sequencing using ionophores, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, and ThermoPhi DNA polymerase. In specific, nonlimiting examples, the polymerase is selected from a group consisting of Bst, Bsu, and Phi29. As the polymerase extends the hybridized strand, it can be beneficial to include single-stranded binding protein (SSB). SSB may stabilize the displaced (non-template) strand. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.” As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “pore” is intended to mean a structure that includes an aperture that permits molecules to cross therethrough from a first side of the pore to a second side of the pore. That is, the aperture extends through the first and second sides of the pore. Molecules that can cross through an aperture of a pore can include, for example, ions or water-soluble molecules such as amino acids or nucleotides. The pore can be disposed within a barrier. When at least a portion of the aperture of a pore has a width of 100 nm or less, e.g., 10 nm or less, or 2 nm or less, the pore can be, but need not necessarily be, referred to as a “nanopore.” Optionally, a portion of the aperture can be narrower than one or both of the first and second sides of the pore, in which case that portion of the aperture can be referred to as a “constriction.” Alternatively or additionally, the aperture of a pore, or the constriction of a pore (if present), or both, can be greater than 0.1 nm, 0.5 nm, 1 nm, 10 nm or more. A pore can include multiple constrictions, e.g., at least two, or three, or four, or five, or more than five constrictions.

As used herein, an “ionophore” is a pore that selectively passes ions therethrough. By “selectively passes ions” it is intended to mean that the ionophore is configured so as to transport ions preferentially to other types of molecules. For example, the dimensions of ionophores are such that the passage of relatively large molecules (such as polynucleotides or proteins) is inhibited. The ions that are selectively passed by an ionophore may be solvated, e.g., may include water molecules coupled to the ions such as via ionic forces or hydrogen bonding, e.g., in a manner such as disclosed in Finkelstein et al., “The gramicidin A channel: a review of its permeability characteristics with special reference to the single-file aspect of transport,” J. Membr. Biol. 59(3): 155-171 (1981), the entire contents of which are incorporated by reference herein. Ionophores may become coupled to (e.g., may insert into) a barrier, and may increase the flow of ions through the barrier when coupled to that barrier relative to the absence of the ionophore. As such, the selective passing of ions through the barrier means that an ionophore is disposed within, or otherwise suitably coupled to, that barrier.

Different types of ionophores may selectively pass different types of ions. For example, some ionophores are selective for positively charged ions, such as protons (H+), sodium (Na+), and/or potassium (K+), while other ionophores are selective for negatively charged ions, such as chloride (Cl−). Nonlimiting examples of ionophores include gramicidin A (gA), gramicidin B (gB), gramicidin C (gC), and fengycin (FE). The sequence of naturally occurring gA, gB, and gC is:

(SEQ ID NO: 1)
formyl-L-X-Gly-L-Ala-D-Leu-L-Ala-D-Val-L-Val-D-
Val-L-Trp-D-Leu-L-Y-D-Leu-L-Trp-D-Leu-L-Trp-
ethanolamine,

where Y is L-tryptophan in gA, Y is L-phenylalanine in gB, and Y is L-tyrosine in gC, and where X determines isoform and is L-valine or L-isoleucine. For further details regarding FE, see Zakharova et al., “Fengycin induces ion channels in lipid bilayers mimicking target fungal cell membranes,” Scientific Reports 9: article number 16034 (2019), the entire contents of which are incorporated by reference herein.

It will be appreciated that use of the term “ionophore” herein, as well as the specific examples provided herein, are intended to encompass any suitable natural or artificial variations. That is, reference to “gA” is intended to encompass natural and artificial variations of the naturally occurring sequence of gA provided above, reference to “gB” is intended to encompass natural and artificial variations of the naturally occurring sequence of gB provided above, reference to “gC” is intended to encompass natural and artificial variations of the naturally occurring sequence of gC provided above, and reference to “FE” is intended to encompass natural and artificial variations of the naturally occurring sequence of FE, which is produced by Bacillus subtilis. For example, an ionophore may be modified so as to pass a particular type of ion(s), to have particular ion conduction characteristics, or so as to be attached to another element. Illustratively, a gA typically passes the positively charged ions Na+ and K+ at a particular flow rate, but may be modified so as to pass these ions at a different flow rate, or may be modified so as to pass negatively charged ions such as Cl−, at a particular flow rate. As another example, a chemical group such as a tert-butyloxycarbonyl (BOC) protected glycine, a sulfonate group, an amine group, or the like may be coupled to the C-terminus of an ionophore in a manner such as described in Capone et al., “Designing nanosensors based on charged derivatives of gramicidin A,” JACS 129: 9737-9745 (2007), the entire contents of which are incorporated by reference herein.

Ionophores such as gA, gB, gC, and FE may dimerize to form a channel that passes ions. Illustratively, ionophores may dimerize with one another through a process in which a first ionophore (e.g., gA, gB, gC, or FE) may be located in a first layer of a lipid bilayer, and a second ionophore (e.g., gA, gB, gC, or FE) may be located in a second layer of the lipid bilayer. The first and second ionophores may become coupled to one another such that the resulting dimer may pass ions across the lipid bilayer, whereas neither the first ionophore alone in the first layer of the lipid bilayer, nor the second ionophore alone in the second layer of the lipid bilayer, nor the lipid bilayer itself, may pass ions (or other molecules) across the lipid bilayer. As used herein, the term “ionophore” may be used to refer to one half of a dimer, or to refer to two dimer halves in contact to form an ion conducting channel through the barrier. When referring to one half of an ionophore, it is understood that even if the half is coupled to the barrier, ions may not pass. As used herein, a “barrier” is intended to mean a structure that normally inhibits passage of molecules from one side of the barrier to the other side of the barrier. The molecules for which passage is inhibited can include, for example, ions or water soluble molecules such as nucleotides and amino acids. However, if a pore is disposed within a barrier, then the aperture of the pore may permit passage of molecules from one side of the barrier to the other side of the barrier. As one specific examples, if an ionophore is disposed within a barrier, the aperture of the ionophore may selectively permit passage of ions from one side of the barrier to the other side of the barrier. Barriers include membranes of biological origin, such as lipid bilayers, and non-biological barriers such as solid state membranes.

As used herein, “linker” is intended to mean an elongated member having a head region, a tail region, and an elongated body therebetween. A linker can include a molecule. A linker can be, but need not necessarily be, in an elongated state, e.g., can include an elongated molecule. For example, an elongated body of a linker can have secondary or tertiary configurations such as hairpins, folds, helical configurations, or the like. Linkers can include polymers such as polynucleotides or synthetic polymers. Linkers can have lengths (e.g., measured in a stretched or maximally extended state) ranging, for example, from about 5 nm to about 500 nm, e.g., from about 10 nm to about 100 nm. Linkers can have widths ranging, for example, from about 1 nm to about 50 nm, e.g., from about 2 nm to about 20 nm. Linkers can be linear or branched. As used herein, a “head region” of a linker is intended to mean a functional group at one end of the linker that is attached to another member, and a “tail region” of a linker is intended to mean a functional group at the other end of the linker that is attached to another member. Such attachments of the head region and tail region respectively can be formed via a chemical bond, e.g., via a covalent bond, hydrogen bond, ionic bond, dipole-dipole bond, London dispersion forces, or any suitable combination thereof. In one example, such attachment can be formed through hybridization of a first oligonucleotide of the head region to a second oligonucleotide of another member. Alternatively, such attachment can be formed using physical or biological interactions, e.g., an interaction between a first protein structure of the head region and a second protein structure of the other member that inhibits detachment of the head region from the other member. Example members to which a head region or a tail region of a linker can be attached include an ionophore, a barrier to which the ionophore coupled, and a molecule, such as a nucleotide or a protein (e.g., membrane spanning protein), disposed on the first and/or second side of the barrier.

As used herein, an “elongated body” is intended to mean a portion of a member, such as a linker, that extends between the head region and the tail region. An elongated body can be formed of any suitable material of biological origin or nonbiological origin, or a combination thereof. In one example, the elongated body includes a polymer. Polymers can be biological or synthetic polymers. Example biological polymers that suitably can be included within an elongated body include polynucleotides, polypeptides, polysaccharides, polynucleotide analogs, and polypeptide analogs. Example polynucleotides and polynucleotide analogs suitable for use in an elongated body include DNA, enantiomeric DNA, RNA, PNA (peptide-nucleic acid), morpholinos, and LNA (locked nucleic acid). Example synthetic polypeptides can include charged amino acids as well as hydrophilic and neutral residues. Example synthetic polymers that suitably can be included within an elongated body include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON™ (tetrafluoroethylene), thermoplastic polyurethanes, polyaldehydes, polyolefins, poly(ethylene oxides), poly(w-alkenoic acid esters), poly(alkyl methacrylates), and other polymeric chemical and biological linkers such as described in Hermanson, Bioconjugate Techniques, third edition, Academic Press, London (2013).

### Polynucleotide Sequencing Using Ionophores

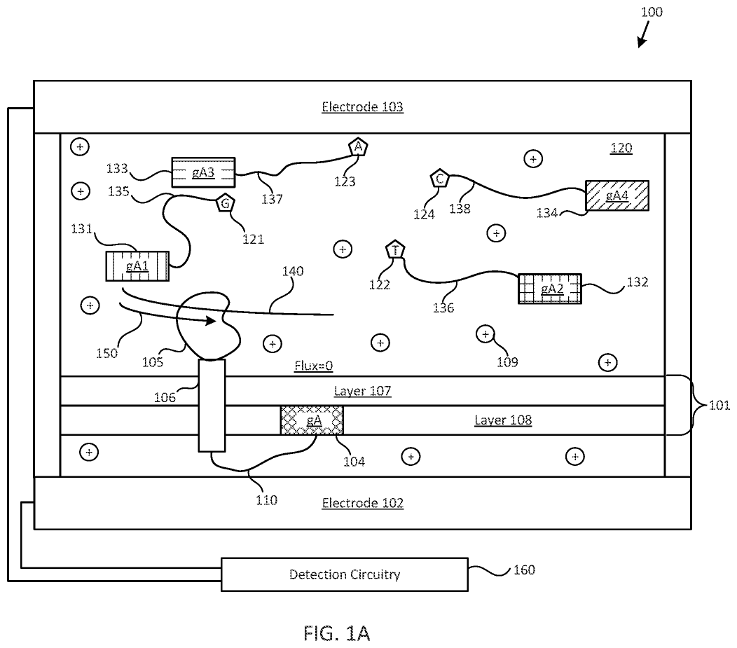

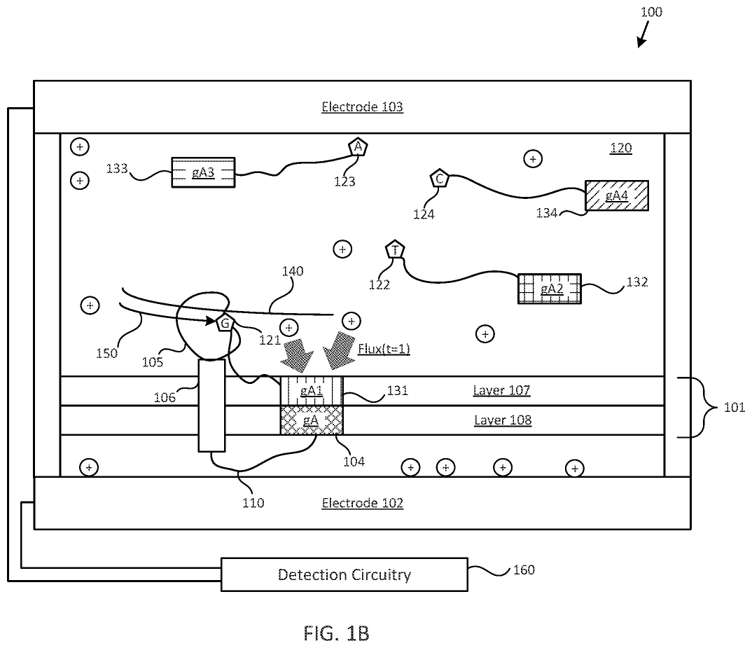

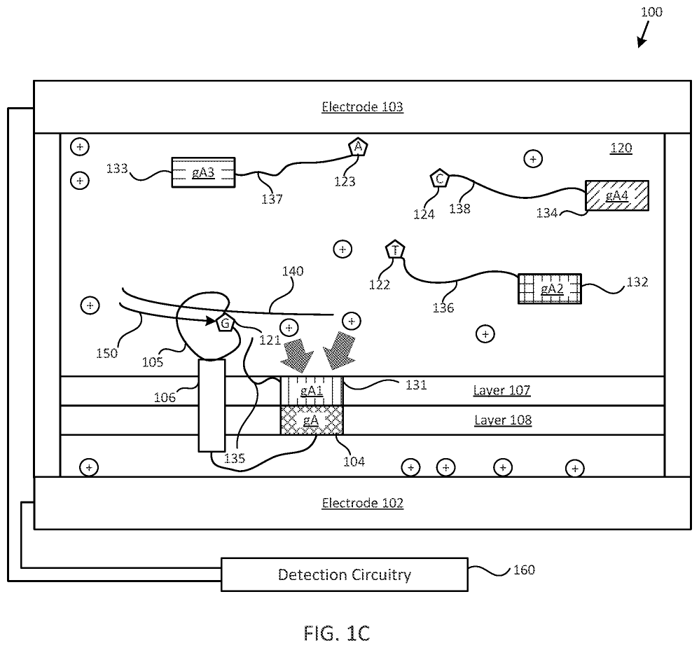

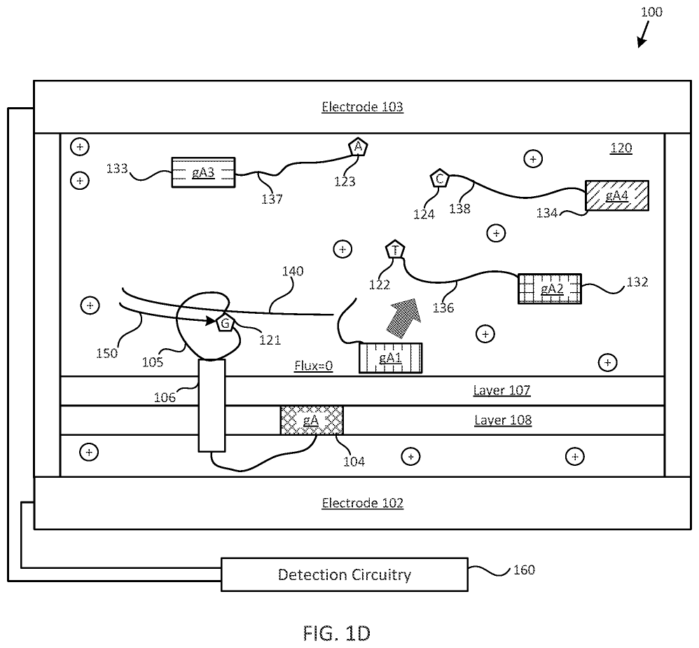

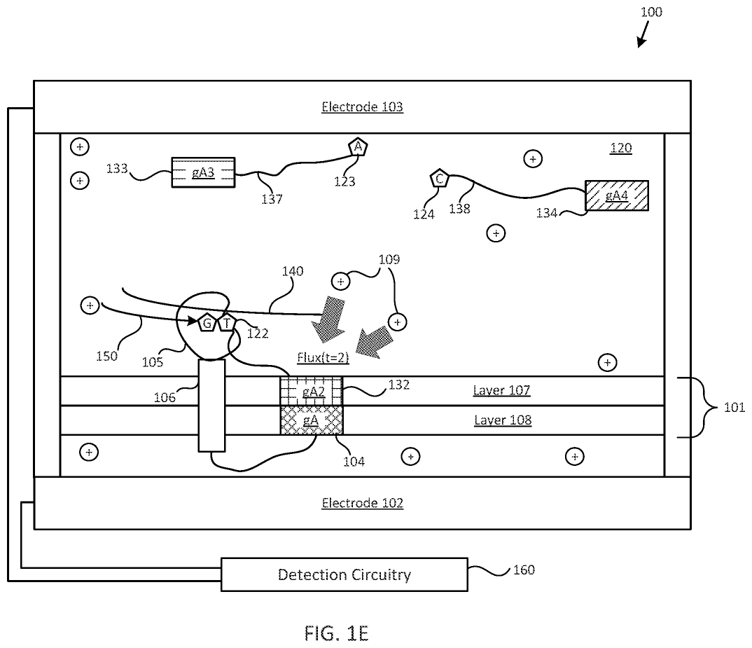

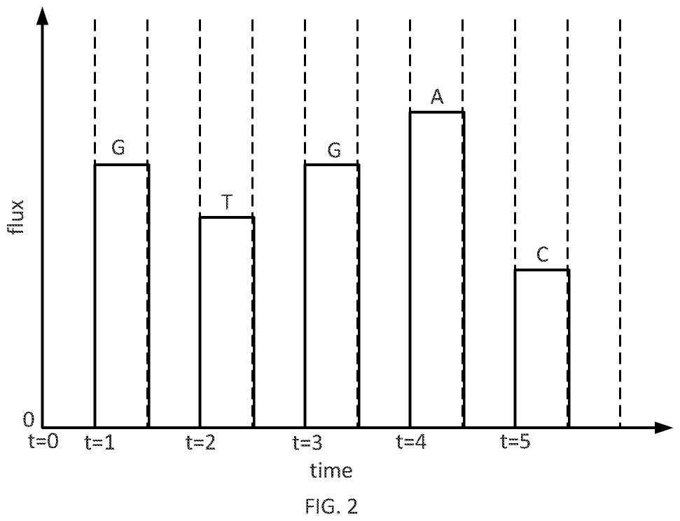

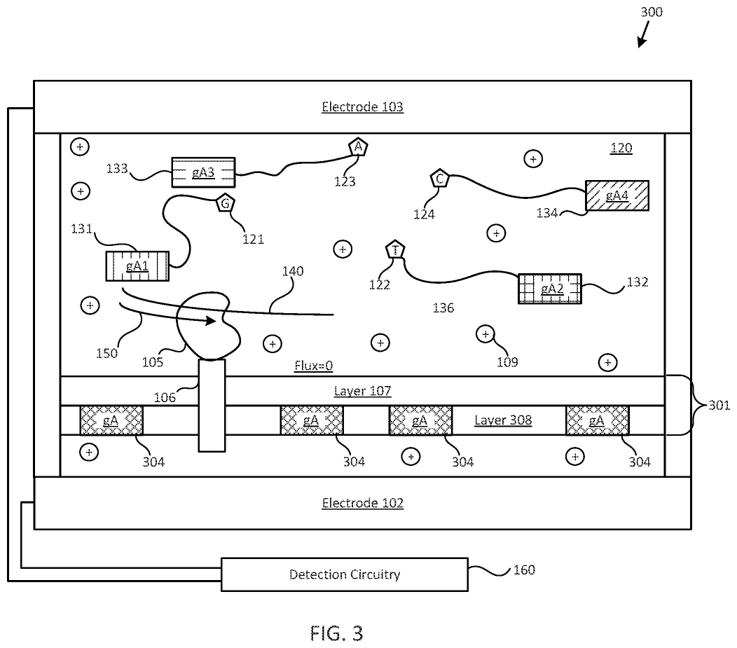

Some example compositions, systems, and methods for polynucleotide sequencing using ionophores now will be described with reference to FIGS. 1A-1E and 2. Other nonlimiting examples will be described with reference to FIGS. 3, 4, 5, 6, and 7.

FIGS. 1A-1E schematically illustrate example compositions and systems for polynucleotide sequencing using ionophores. System 100 illustrated in FIG. 1A includes a composition that includes barrier 101, first electrode 102, second electrode 103, polymerase 105, fluid 120 including a nucleotide coupled to an ionophore, first polynucleotide 140, and second polynucleotide 150; and detection circuitry 160. Polymerase 105 may be in contact with first polynucleotide 140, second polynucleotide 150, and fluid 120. Polymerase 105 optionally may be coupled to barrier 101 in any suitable manner, such as via optional membrane spanning protein (MSP) 106.

Barrier 101 may have any suitable structure that normally inhibits current flow across the barrier. For example, as illustrated in FIG. 1A, barrier 101 may include first layer 107 and second layer 108, one or both of which inhibit the flow of ions, and thus of current, across that layer. At the particular time illustrated in FIG. 1A, layer 108 includes ionophore 104 which may be coupled to polymerase 105 via MSP 106, while layer 107 lacks any ionophore. As such, although ionophore 104 may permit ionic flow (and thus current flow) across layer 108, the absence of any ionophore within layer 107 inhibits ionic flow (and thus current flow) across layer 107 and thus across barrier 101. Detection circuitry 160 is configured to measure an electrical characteristic of barrier 101 that may arise from the flow of current across barrier 101 and between electrode 102 and electrode 103, e.g., a magnitude or temporal duration of a voltage, current, or resistance. At the particular time illustrated in FIG. 1A (t=0), because the flow of current across barrier 101 is inhibited, detection circuitry 160 may measure a current of approximately zero, as indicated by “Flux=0” in FIG. 1A, or other suitable electrical characteristic.

In the nonlimiting example illustrated in FIG. 1A, fluid 120 may include ions 109 (e.g., cations as indicated by “+” in FIG. 1A, or anions). Fluid 120 also may include a first nucleotide analogue including first nucleotide 121 (e.g., G) coupled to first ionophore 131 via linker 135; a second nucleotide analogue including second nucleotide 122 (e.g., T) coupled to second ionophore 132 via linker 136; a third nucleotide analogue including third nucleotide 123 (e.g., A) coupled to third ionophore 133 via linker 137; and a fourth nucleotide analogue including fourth nucleotide 124 (e.g., C) coupled to fourth ionophore 134 via linker 138. Illustratively, ionophores 131, 132, 133, 134 of the nucleotide analogues each may include a respective gramicidin A (respectively referred to as gA1, gA2, gA3, and gA4 in FIG. 1A), and ionophore 104 within barrier 101 may include another gA. However, although gA is illustrated for simplicity, it will be appreciated that any suitable ionophore(s) may be coupled to respective nucleotides 121, 122, 123, 124 and to barrier 101. For example, the nucleotides and barrier respectively may be coupled to any suitable combinations of gA, gB, gC, or fengycin. For example, the nucleotides may be coupled to differently modified ionophores of a given type, and the barrier may be coupled to an ionophore of that type which is suitable to dimerize with each of the ionophores coupled to the respective nucleotides. The ionophores of the nucleotide analogues may provide flow of ions 109 across barrier 101, and thus may provide respective non-zero currents across barrier 101. As such, detection circuitry 160 may be configured to determine the sequence of first polynucleotide 140 using electrical characterizations of the different ionophores corresponding the nucleotides as the nucleotides are added to second polynucleotide 150. Note, however, that in some examples the ionophores may not pass nucleotides or polynucleotides, and indeed may be limited to pass only ions 109 (e.g., cations or anions). The ionophores (such as gA1, gA2, gA3, and gA4) of the nucleotide analogues may differ from one another in such a manner as to pass different fluxes of ions 109 and thus to provide measurably different currents across barrier 101 from which the identity of the corresponding nucleotide may be identified, e.g., in a manner such as will now be described with reference to FIGS. 1B-1E.

For example, as illustrated in FIG. 1B, polymerase 105 may add a nucleotide to second polynucleotide 150 based on a sequence of first polynucleotide 140. Illustratively, the next base in first polynucleotide 140 may be C, based upon which polymerase 105 may add first nucleotide 121 (G) to second polynucleotide 150. First ionophore 131, which is coupled to first nucleotide 121, may provide a first current flow across the barrier, and may be electrically characterized using detection circuitry 160. For example, in a manner such as described with reference to FIG. 1A, barrier 101 may include ionophore 104. As illustrated in FIG. 1B, ionophore 131 may become coupled to ionophore 104 to provide the first current flow across the barrier, as indicated by the shaded arrows suggesting movement of ions 109 (e.g., cations or anions) toward and through ionophores 131 and 104. At the particular time illustrated in FIG. 1B (t=1), the rate of the flow of ions (and thus the current flow) across barrier 101 is determined by the particular characteristics of ionophore 131, and detection circuitry 160 may measure a corresponding nonzero current across barrier 101 at this time (“Flux(t=1)”), or other suitable electrical characteristic.

Illustratively, barrier 101 may include an electrical insulator through which first ionophore 131 forms an aperture that provides the first current flow across the barrier. For example, barrier 101 may include a membrane, such as a lipid bilayer (one layer of which corresponds to layer 107 illustrated in FIG. 1B, and the other layer of which corresponds to layer 108 illustrated in FIG. 1B). Ionophore 131 may insert into a layer of the lipid bilayer, e.g., into layer 107. The membrane, e.g., layer 107, may include ionophore 104 to which each of the respective ionophores 131, 132, 133, 134 is couplable. For example, ionophore 131 may insert into layer 107 and be held in proximity of polymerase 105 by linker 135. Similarly, ionophore 104 may be inserted into layer 107 and be held in proximity of polymerase 105 by linker 110. Because ionophores 104 and 131 are both held in proximity of polymerase 105, they may be kinetically more likely to remain coupled to one another long enough to be detectable by detection circuitry 160 than are other pairs of ionophores for which any coupling is likely to be relatively brief.

Ionophore 131 (in addition to ionophores 132, 133, and 134) may be detachable, by polymerase 105, from first nucleotide 121 so as to diffuse away from barrier 101 so as to again inhibit current flow across the barrier. Additionally, ionophore 131 may be detachable from the second ionophore so as to inhibit current flow across the barrier. For example, in a manner such as illustrated in FIG. 1C, after adding nucleotide 121 to polymerase 105 may cleave linker 135 so as to detach ionophore 131 from nucleotide 121. Ionophore 131 may remain coupled to barrier 101 and/or to second ionophore for a period of time such that ions may continue to pass therethrough, as illustrated in FIG. 1C. However, ionophore 131 eventually may diffuse away from or otherwise become detached from barrier 101 and from ionophore 104 in a manner such as suggested by the shaded arrow illustrated in FIG. 1D.

After polymerase 105 cleaves linker 135 from nucleotide 121, the polymerase may add another nucleotide to second polynucleotide 150 based on a sequence of first polynucleotide 140. Illustratively, the next base in first polynucleotide 140 may be A, based upon which polymerase 105 may add second nucleotide 122 (T) to second polynucleotide 150 in a manner such as illustrated in FIG. 1E. Second ionophore 132, which is coupled to second nucleotide 122, may provide a second current flow across the barrier in a manner similar to that described with reference to FIG. 1B. For example, ionophore 132 may become coupled to ionophore 104 to provide the second current flow across the barrier, as indicated by the shaded arrows suggesting movement of ions 109 (e.g., cations or anions) toward and through ionophores 132 and 104. At the particular time illustrated in FIG. 1E (t=2), the rate of the flow of ions (and thus the current flow) across barrier 101 is determined by the particular characteristics of ionophore 132, and detection circuitry 160 may measure a corresponding nonzero current across barrier 101 at this time (“Flux(t=2)”), or other suitable electrical characteristic. Ionophore 132 may be detached from barrier 101 and from ionophore 104 in a manner similar to that described with reference to FIGS. 1C-1D, and polymerase 105 may add additional nucleotides to second polynucleotide 150 in a similar manner as described for nucleotides 121 and 122.

As noted elsewhere herein, at least some of the respective ionophores 131, 132, 133, and/or 134 may have different modifications than one another, and as a result may pass ions at different rates than one another. As such, these ionophores may have different electrical characteristics based upon which the nucleotides may be identified to which such ionophores respectively are coupled. For example, FIG. 2 schematically illustrates example signals that may be obtained during polynucleotide sequencing using the system of FIGS. 1A-1E. It will be appreciated that the particular sequence in which nucleotides are added is for illustrative purposes, and that although FIG. 2 may appear to suggest that the coupling of ionophores to barrier 101 and the detachment of ionophores from barrier 101 happen at regular intervals, the actual timing of such coupling and detachment may vary significantly.

At an initial time (t=0, such as illustrated in FIG. 1A), substantially no current flows across barrier 101 and as such detection circuitry 160 may measure a flux of about zero, or other suitable electrical characteristic. At a subsequent time (t=1, such as illustrated in FIG. 1B), responsive to polymerase 105 acting upon nucleotide 121, ionophore 131 provides a first current flow across barrier 101, resulting in a first flux value corresponding to nucleotide 121 (e.g., G). After ionophore 131 detaches from barrier 101 (e.g., after polymerase 105 adds nucleotide 121 to second polynucleotide 150), the flux returns to about zero. At a subsequent time (t=2, such as illustrated in FIG. 1E), responsive to polymerase 105 acting upon nucleotide 122, ionophore 132 provides a second current flow across barrier 101, resulting in a second flux value corresponding to nucleotide 122 (e.g., T). After ionophore 132 detaches from barrier 101 (e.g., after polymerase 105 adds nucleotide 122 to second polynucleotide 150), the flux returns to about zero. At a subsequent time (t=3), responsive to polymerase 105 acting upon another nucleotide 121, ionophore 131 provides a first current flow across barrier 101, resulting in a first flux value corresponding to nucleotide 121 (e.g., G). Note that the first flux values measured at t=1 and t=3 are about the same as one another, because both of nucleotides 121 were coupled to a respective ionophore 131 with the same properties for passing ions.

After ionophore 131 detaches from barrier 101 (e.g., after polymerase 105 adds nucleotide 121 to second polynucleotide 150), the flux returns to about zero. At a subsequent time (t=4), responsive to polymerase 105 acting upon nucleotide 123, ionophore 133 provides a third current flow across barrier 101, resulting in a third flux value corresponding to nucleotide 123 (e.g., A). After ionophore 133 detaches from barrier 101 (e.g., after polymerase 105 adds nucleotide 123 to second polynucleotide 150), the flux returns to about zero. At a subsequent time (t=5), responsive to polymerase 105 acting upon nucleotide 124, ionophore 134 provides a fourth current flow across barrier 101, resulting in a fourth flux value corresponding to nucleotide 124 (e.g., C). After ionophore 134 detaches from barrier 101 (e.g., after polymerase 105 adds nucleotide 124 to second polynucleotide 150), the flux returns to about zero. Thus, it may be understood that detection circuitry 160 may measure a flux of about zero between the addition of nucleotides, and may measure fluxes with values that may correspond to the particular ionophores to which nucleotides are coupled. As such, the nucleotides may be identified using such fluxes. It will be appreciated, however, that flux is just one of many different electrical characteristics that detection circuitry 160 may measure. Additionally, although for simplicity, FIG. 2 illustrates an electrical measurement including evenly spaced ionophore insertion events with equal durations, it will be appreciated that different ionophores may be used that have different temporal durations, e.g., different kinetics (such as on-off rates) arising from differences between the ionophores. Detection circuitry 160 may be configured to measure any suitable combination of temporal duration and/or magnitude of voltage, current, or resistance through the ionophores, and to identify the nucleotides based thereon.

It will be appreciated that fluid 120 described with reference to FIGS. 1A-1E may include any suitable combination of nucleotide analogues, ions, buffers, solvents, and the like. In some examples, fluid 120 may include at least one nucleotide analogue. Each of the nucleotide analogues may include a sugar, a nucleobase, a phosphate group, and an ionophore. The nucleobase (e.g., pyrimidine or purine) and phosphate group may be coupled to the sugar in a standard fashion, and the ionophore may be indirectly coupled to the sugar via the phosphate group. For example, the nucleotides may have the structure:

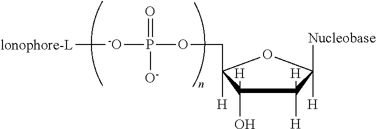

where n is greater than one (e.g., is 2, 3, 4, 5, 6, or greater than 6), and where L represents an optional linker coupling the ionophore to the phosphate group. For example, the phosphate group may be selected from the group consisting of triphosphate, tetraphosphate, pentaphosphate, and hexaphosphate. Example linkers are described elsewhere herein. In a manner such as described with reference to FIGS. 1A-1E, in various examples the ionophore may include gA, gB, gC, or fengycin, and/or may selectively pass anions or cations. Within fluid 120, the ionophores of a first set of the nucleotide analogues may be different than the ionophores of a second set of the nucleotide analogues, e.g., may include a modification that the second set does not include. Illustratively, in a manner such as described with reference to FIG. 2, the ionophores of the first set of nucleotide analogues may have a first electrical characteristic, and the ionophores of the second set of the nucleotide analogues may have a second electrical characteristic that is different than the first electrical characteristic. Similarly, the ionophores of a third set of nucleotide analogues may have a third electrical characteristic that is different than the first and second electrical characteristics, and the ionophores of a fourth set of the nucleotide analogues may have a fourth electrical characteristic that is different than the first, second, and third electrical characteristics. As such, the different electrical characteristics may result in detection circuitry 160 measuring different values that may be used to identify the different nucleotides being added to the growing primer (150) in a sequence complementary to that of the template polynucleotide (140). As noted elsewhere herein, detection circuitry 160 may measure a magnitude and/or temporal duration of a current, resistance, or voltage through the respective ionophore.

It will be appreciated that compositions, systems, and operations such as described with reference to FIGS. 1A-1E suitably may be modified. For example, different types or numbers of ionophores may be provided within the barriers in a manner so as to alter the kinetics of ionophore insertion into, and diffusion away from, the barrier. Illustratively, FIG. 3 schematically illustrates an alternative example composition and system for polynucleotide sequencing using ionophores. System 300 illustrated in FIG. 3 includes a composition that includes barrier 301, first electrode 102, second electrode 103, polymerase 105, fluid 120 including ions 109 and nucleotides 121, 122, 123, 124 respectively coupled to ionophores 131, 132, 133, 134, first polynucleotide 140, and second polynucleotide 150; and detection circuitry 160. In a manner similar to that described with reference to FIGS. 1A-1E, polymerase 105 may be in contact with first polynucleotide 140, second polynucleotide 150, and fluid 120, and optionally may be coupled to barrier 301 in any suitable manner, such as via optional membrane spanning protein (MSP) 106. Similarly as barrier 101, barrier 301 may have any suitable structure that normally inhibits current flow across the barrier, and may include first layer 107 and second layer 308, one or both of which inhibit the flow of ions, and thus of current, across that layer. Illustratively, barrier 101 may include a membrane, such as a lipid bilayer of which layer 107 forms one layer and second layer 308 forms the other layer.

In a manner such as illustrated in FIG. 3, second layer 308 of barrier 301 may include a plurality of ionophores 304, each of which may be configured similarly as ionophore 104. For example, ionophores 304 may have substantially the same configuration as one another, and optionally may be coupled to the polymerase 105, e.g., via optional MSP 106 and/or via optional linkers (linkers not specifically illustrated in FIG. 3). At the particular time illustrated in FIG. 3 (t=0), the flow of current across barrier 301 is inhibited, and as such detection circuitry 160 may measure a current of substantially zero, as indicated by “Flux=0” in FIG. 3, or other suitable electrical characteristic, in a manner similar to that described with reference to FIG. 1A. Based on the sequence of first polynucleotide 140, polymerase 105 may add nucleotides 121, 122, 123, and 124 to second polynucleotide 150, responsive to which ionophores 131, 132, 133, 134 may become coupled to ionophores 304 within barrier 301 in such a manner as to provide respective currents therethrough, based upon which the nucleotides may be identified in a manner such as described with reference to FIGS. 1B-1E.

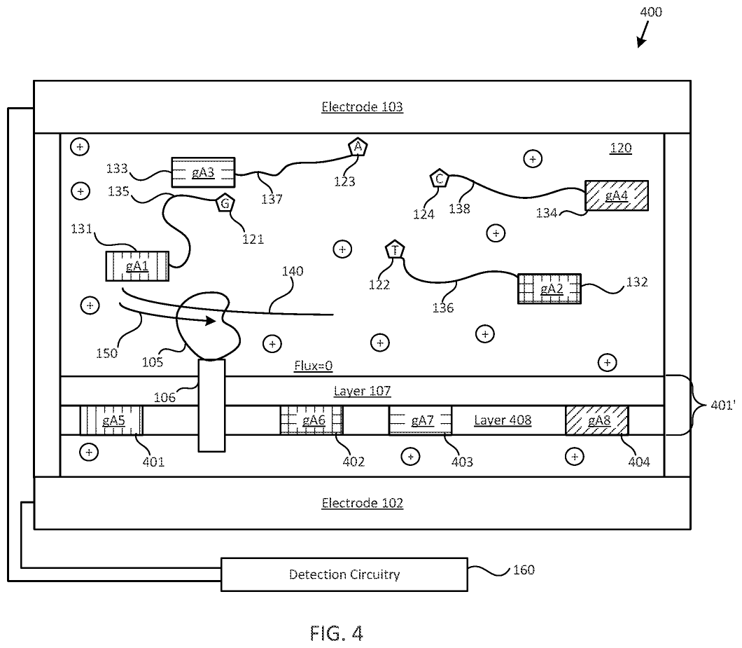

As another example, FIG. 4 schematically illustrates an alternative example composition and system for polynucleotide sequencing using ionophores. System 400 illustrated in FIG. 4 includes a composition that includes barrier 401′, first electrode 102, second electrode 103, polymerase 105, fluid 120 including ions 109 and nucleotides 121, 122, 123, 124 respectively coupled to ionophores 131, 132, 133, 134, first polynucleotide 140, and second polynucleotide 150; and detection circuitry 160. In a manner similar to that described with reference to FIGS. 1A-1E, polymerase 105 may be in contact with first polynucleotide 140, second polynucleotide 150, and fluid 120, and optionally may be coupled to barrier 401′ in any suitable manner, such as via optional membrane spanning protein (MSP) 106. Similarly as barrier 101, barrier 401′ may have any suitable structure that normally inhibits current flow across the barrier, and may include first layer 107 and second layer 408, one or both of which inhibit the flow of ions, and thus of current, across that layer. Illustratively, barrier 101 may include a membrane, such as a lipid bilayer of which layer 107 forms one layer and second layer 408 forms the other layer.

In a manner such as illustrated in FIG. 4, second layer 408 of barrier 401′ may include a plurality of ionophores 401, 402, 403, 404 to which the respective ionophores are selectively couplable. For example, ionophores 401, 402, 403, 404 may be configured differently than one another, such that ionophore 401 (e.g., gA5) couples to ionophore 131 but not to any of ionophores 132, 133, or 134; ionophore 402 (e.g., gA6) couples to ionophore 132 but not to any of ionophores 131, 133, or 134; ionophore 403 (e.g., gA7) couples to ionophore 133 but not to any of ionophores 131, 132, or 134; and ionophore 404 (e.g., gA8) couples to ionophore 134 but not to any of ionophores 131, 132, or 133. Ionophores 401, 402, 403, 404 optionally may be coupled to polymerase 105, e.g., via optional MSP 106 and/or via linkers (linkers not specifically illustrated in FIG. 4). At the particular time illustrated in FIG. 4 (t=0), the flow of current across barrier 401′ is inhibited, and as such detection circuitry 160 may measure a current of substantially zero, as indicated by “Flux=0” in FIG. 3, or other suitable electrical characteristic, in a manner similar to that described with reference to FIG. 1A. Based on the sequence of first polynucleotide 140, polymerase 105 may add nucleotides 121, 122, 123, and 124 to second polynucleotide 150, responsive to which ionophores 131, 132, 133, 134 respectively may become coupled to ionophores 401, 402, 403, 404 within barrier 401 in such a manner as to provide respective currents therethrough. The nucleotides may be identified using electrical characterizations of the ionophores in a manner such as described with reference to FIGS. 1B-1E.

Note that any suitable electrical circuitry may be used to make a measurement relating to the flow of ions, via ionophores, through barriers that otherwise inhibit such flow. For example, detection circuitry 160 described with reference to FIGS. 1A-1E, 3, and 4 may include electrical circuitry for identifying nucleotides 121, 122, 123, 124 using the magnitude and/or temporal duration of the respective current flow, resistance, or voltage through ionophores 131, 132, 133, 134. In some examples, such measurement may utilize non-Faradaic current. For example, in non-Faradaic current, substantially no electrons may be transferred between the fluid 120 and electrodes 102 and 103. Detection circuitry 160 may apply an electric field that forces ions 109 to move within fluid 120 in such a manner as to aggregate at electrode 102 in a manner such as illustrated in FIG. 1B while any negatively charged ions aggregate at electrode 103 (or vice versa). In examples in which ions 109 are cations (such as K+ and/or Na+), their aggregation at one electrode electrostatically attracts electrons to that electrode (e.g., electrode 102), while the aggregation of any negatively charged ions at the other electrode (e.g., electrode 103) electrostatically repels electrons from that electrode. Such aggregations of ions generate a potential difference between electrodes 102 and 103 that drive transient currents through detection circuitry 160 (e.g., an external electric circuit) that are measured and that persist until the potential difference between electrode 102 and 103 is diminished to a level that no longer drives current. Because ionophores 131, 132, 133, 134 conduct ions differently than one another, such ionophores may generate different aggregations of ions at electrodes 102 and/or 103 which in turn generate different transient currents that may be used to identify the nucleotides 121, 122, 123, 124 to which those ionophores respectively are coupled.

In some examples, detection circuitry 160 may reverse the electric field once or repeatedly, so as to alternate the electrodes at which ions 109 aggregate, and then to use the resulting transient circuits to identify the nucleotides in a similar manner. As such, the use of an alternating electric field may be considered to repeatedly charge and discharge a capacitor formed by electrodes 102 and 103. Detection circuitry 160 may alternate the electric field at a frequency that is sufficiently high to inhibit saturation of such capacitor, and sufficiently low to permit accurate sensing of current flow through each ionophore that becomes coupled to the barrier. For example, a flow of about 1,000 ions per millisecond may be expected through each ionophore. The total number of ions that flow per nucleotide incorporation event may be expected to be a function of at least the time the nucleotide 121, 122, 123, 124 spends in the active site of polymerase 105 before cleavage of the respective ionophore 131, 132, 133, 134, and the stability of the dimer formed between that ionophore and ionophore 104, 304, 401, 402, 403, or 404. Additionally, the size of electrodes 102, 103 may be suitably selected to accommodate sufficient current flow. As one purely illustrative example, electrodes 102, 103 each may have an area of about 10 μm² to about 100 μm². Typical capacitances of two-electrode capacitors are about 10 μF/cm² to about 0.1 pF/cm², or about 1-10 pF for example electrodes having an area of about 10 μm² to about 100 μm². Based upon detection circuitry 160 alternating the electric field at an example frequency of 10 kHz with 10 mV amplitude, a dV/dt of about 100 V/s would be generated which would generate a displacement current of about 10 pA/μm², or a total current of about 0.1-1.0 nA. It is expected that such current would be readily measurable using a high precision amplifier, for example using frequencies of about 10-100 kHz alternating current.

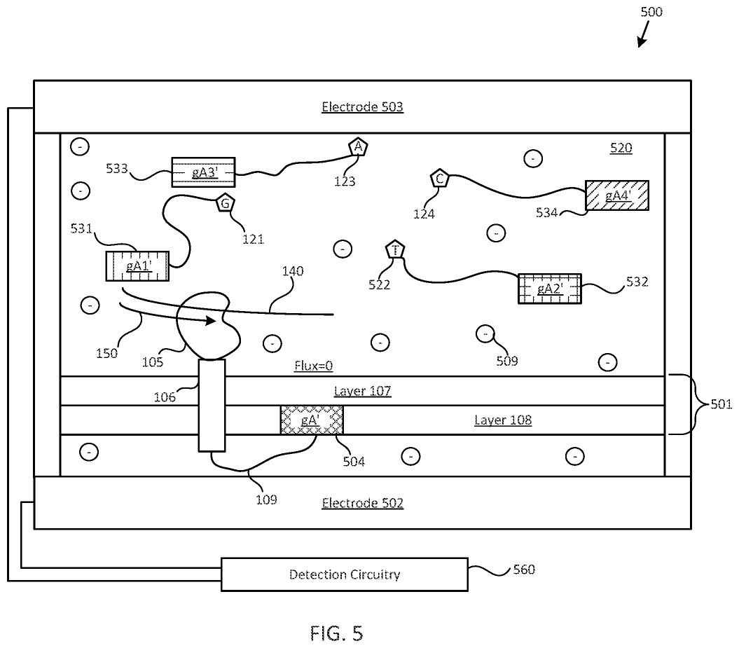

Alternatively, the detection circuitry 160 may utilize Faradaic current to identify nucleotides. For example, FIG. 5 schematically illustrates another alternative example composition and system for polynucleotide sequencing using ionophores. System 500 illustrated in FIG. 5 includes a composition that includes barrier 501, first electrode 502, second electrode 503, polymerase 105, fluid 520 including ions 509 and nucleotides 121, 122, 123, 124 respectively coupled to ionophores 531, 532, 533, 534 (e.g., gA1′, gA2′, gA3′, ga4′), first polynucleotide 140, and second polynucleotide 150; and detection circuitry 560. In a manner similar to that described with reference to FIGS. 1A-1E, polymerase 105 may be in contact with first polynucleotide 140, second polynucleotide 150, and fluid 520, and optionally may be coupled to barrier 501 in any suitable manner, such as via optional membrane spanning protein (MSP) 106. Similarly as barrier 101, barrier 501 may have any suitable structure that normally inhibits current flow across the barrier, and may include first layer 107 and second layer 508, one or both of which inhibit the flow of ions, and thus of current, across that layer. Illustratively, barrier 501 may include a membrane, such as a lipid bilayer of which layer 107 forms one layer and second layer 508 forms the other layer.

In a manner such as illustrated in FIG. 5, second layer 508 of barrier 501 may include ionophore 504 (e.g., gA′), which may be configured similarly as ionophore 104, optionally may be coupled to MSP 106 via linker 110, and may be configured so as to selectively pass anions 509. Similarly, ionophores 531, 532, 533, 534 may be configured similarly as ionophores 131, 132, 133, 134 and may be configured so as to selectively pass anions 509. At the particular time illustrated in FIG. 5 (t=0), the flow of current across barrier 501 is inhibited, and as such detection circuitry 560 may measure a current of substantially zero, as indicated by “Flux=0” in FIG. 5, or other suitable electrical characteristic, in a manner similar to that described with reference to FIG. 1A. Based on the sequence of first polynucleotide 140, polymerase 105 may add nucleotides 121, 122, 123, and 124 to second polynucleotide 150, responsive to which ionophores 531, 532, 533, 534 may become coupled to ionophore 504 within barrier 501 in such a manner as to provide respective currents therethrough. The nucleotides may be identified using electrical characterizations of the ionophores in a manner such as described with reference to FIGS. 1B-1E.

Whereas detection circuitry 160 may use non-Faradaic current in a manner such as described with reference to FIGS. 1A-1E, detection circuitry 560 may use Faradaic current in which the detection circuitry uses a redox reaction to transfer, to electrode 502 or electrode 503, electrons from ions 509 (e.g., from anions, such as Cl−) that are passed by ionophores 531, 532, 533, 534. Illustratively, electrodes 502 and 503 may include silver (Ag) which reacts with Cl− ions 509 to form silver chloride (AgCl) and generate a free electron that provides a measurable current through detection circuitry 560. It will be appreciated that other ions, and other redox reactions, suitably may be used.

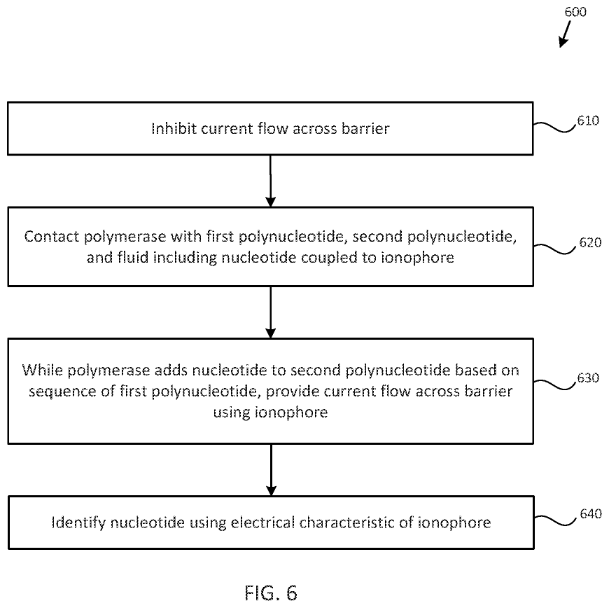

It will be appreciated that systems, compositions, and operations such as described with reference to FIGS. 1A-1E, 2, 3, 4, and 5 suitably may be adapted for use in various methods of polynucleotide sequencing. For example, FIG. 6 illustrates a flow of operations in an example method 600 for polynucleotide sequencing using ionophores. Method 600 may include inhibiting current flow across a barrier (operation 610). For example, in the absence of an ionophore coupled thereto, barrier 101, 301, 401, and 501 may inhibit the flow of ions or other molecules across the barrier, and as such may inhibit current flow across the barrier. Method 600 also may include contacting a polymerase with a first polynucleotide, a second polynucleotide, and a fluid including a nucleotide coupled to an ionophore (operation 620). For example, polymerase 105 may be contacted with first polynucleotide 140, second polynucleotide 150, and fluid 120 including nucleotide 121, 122, 123, 124 coupled to ionophore 131, 132, 133, 134 or fluid 520 including nucleotide 121, 122, 123, 124 to ionophore 531, 532, 533, 534. Method 600 also may include, while the polymerase adds the nucleotide to the second polynucleotide based on a sequence of the first polynucleotide, providing a current flow across the barrier using the ionophore (operation 630). For example, while polymerase 105 adds each nucleotide 121, 122, 123, 124 to second polynucleotide 150 based on the sequence of first polynucleotide 140, ionophore 131, 132, 133, 134 may become coupled to barrier 101, or ionophore 531, 532, 533, 534 may become coupled to barrier 501, in such a manner as to provide a current flow across the barrier. Method 600 may include identifying the nucleotide using an electrical characteristic of the ionophore. For example, each ionophore 131, 132, 133, 134, 531, 532, 533, or 534 may pass ions with a rate that differs from that of the other ionophores. Accordingly, each such ionophore may have a different electrical characteristic based upon which the corresponding nucleotide may be identified in a manner such as described elsewhere herein.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1-6. (canceled)

7. A fluid comprising a plurality of nucleotide analogues, each of the nucleotide analogues comprising:
a sugar;
a nucleobase coupled to the sugar;
a phosphate group coupled to the sugar; and
an ionophore indirectly coupled to the sugar via the phosphate group,
wherein the ionophores of a first set of the nucleotide analogues are different than the ionophores of a second set of the nucleotide analogues, and
wherein the ionophores of the first set of nucleotide analogues have a first electrical characteristic, and wherein the ionophores of the second set of the nucleotide analogues have a second electrical characteristic that is different than the first electrical characteristic.

8. The fluid of claim 7, wherein the ionophores comprise gramicidin A, gramicidin B, gramicidin C, or fengycin.

9-11. (canceled)

12. The fluid of claim 7, wherein the first or second, electrical characteristic comprises a magnitude of a current, resistance, or voltage through the respective ionophore.

13. The fluid of claim 7, wherein the first or second, electrical characteristic comprises a temporal duration of a current through the respective ionophore.

14. A sequencing method, comprising:
inhibiting current flow across a barrier;
contacting a polymerase with a first polynucleotide, a second polynucleotide, and a fluid comprising a first nucleotide coupled to a first ionophore;
while the polymerase adds the first nucleotide to the second polynucleotide based on a sequence of the first polynucleotide, providing a first current flow across the barrier using the first ionophore; and
identifying the first nucleotide using an electrical characteristic of the first ionophore.

15. The method of claim 14, wherein the electrical characteristic comprises a magnitude of the first current flow, a resistance through the first ionophore, or a voltage through the first ionophore.

16. The method of claim 14, wherein the electrical characteristic comprises a temporal duration of the first current flow.

17. (canceled)

18. The method of claim 14, further comprising:
decoupling, using the polymerase, the first ionophore from the first nucleotide; and
diffusing the decoupled first ionophore away from the barrier so as to inhibit current flow across the barrier.

19. The method of claim 14, wherein the barrier comprises a second ionophore to which the first ionophore becomes coupled to provide the first current flow across the barrier.

20. (canceled)

21. The method of claim 19, further comprising decoupling the first ionophore from the second ionophore so as to inhibit current flow across the barrier again.

22. The method of claim 19, wherein the first and second ionophores are selected from the group consisting of gramicidin A, gramicidin B, gramicidin C, and fengycin.

23-26. (canceled)

27. The method of claim 14, wherein the barrier comprises an electrical insulator through which the first ionophore forms an aperture.

28-29. (canceled)

30. The method of claim 14, wherein the barrier comprises a bilayer membrane, and wherein the first ionophore inserts into a layer of the bilayer membrane.

31. The method of claim 14, wherein the fluid further comprises a plurality of additional nucleotides each coupled to a respective ionophore, the method further comprising:
while the polymerase sequentially adds the each of the additional nucleotides to the second polynucleotide based on a sequence of the first polynucleotide, sequentially providing additional current flows across the barrier using the respective ionophore; and
identifying the additional nucleotides using additional electrical characteristics of the additional ionophores.

32-34. (canceled)

35. The method of claim 31, wherein the barrier comprises at least one second ionophore to which each of the respective ionophores become coupled to respectively provide the additional current flows across the barrier.

36. (canceled)

37. The method of claim 14, wherein identifying the first nucleotide comprises transferring to an electrode, using a redox reaction, electrons from ions passed by the first ionophore.

38. (canceled)

39. The method of claim 14, wherein identifying the first nucleotide comprises:
applying a first electric field to generate, at a first electrode, a first aggregation of ions passed by the first ionophore; and
using the first aggregation of ions to generate a first transient current through an external circuit.

40. (canceled)

41. The method of claim 39, further comprising:
applying a second electric field to generate, at a second electrode opposite the first electrode, a second aggregation of ions passed by the first ionophore; and
using the second aggregation of ions to generate a second transient current through the external circuit.

42. (canceled)

43. A composition, comprising:
a barrier;
a polymerase in contact with a first polynucleotide, a second polynucleotide, and a fluid comprising a first nucleotide coupled to a first ionophore,
the polymerase adding the first nucleotide to the second polynucleotide based on a sequence of the first polynucleotide,
the first ionophore providing a first current flow across the barrier.

44-61. (canceled)

62. A system, comprising:
the composition of claim 43; and
electrical circuitry for identifying the first nucleotide using an electrical characteristic of the first ionophore.

63-70. (canceled)


SEQUENCE LISTING (1 sequence)

SEQ ID NO: 1 - protein, 16 aa, Artificial Sequence
  synthetic construct
  MOD_RES (1)..(1): Xaa = formyl-L-valine or formyl-L-isoleucine
  MOD_RES (4)..(4): Xaa = D-Leucine
  MOD_RES (6)..(6): Xaa = D-Valine
  MOD_RES (8)..(8): Xaa = D-Valine
  MOD_RES (10)..(10): Xaa = D-Leucine
  MOD_RES (11)..(11): Xaa = L-tryptophan or L-phenylalanine, or L-tyrosine
  MOD_RES (12)..(12): Xaa = D-Leucine
  MOD_RES (14)..(14): Xaa = D-Leucine
  MOD_RES (16)..(16): Xaa = ethanolamine
XGAXAXVXWXXXWXWX